(19) United States
(12) Patent Application Publication
(10) Pub. No.: US 2009/0287085 A1
(43) Pub. Date: Nov. 19, 2009

(54) Device, system, and method of determining an acoustic contact between an ultrasonic transducer and a body

(76) Inventors: Shmuel Ben-Ezra, Pardes-Hauna (IL); Yehuda Zadok, Holon (IL)

Correspondence Address: EITAN MEHULAL LAW GROUP, 10 Abba Eban Blvd. PO Box 2081, Herzlia, 46120, IL

(21) Appl. No.: 12/153,197

(22) Filed: May 15, 2008

(51) Int. Cl. A61B 8/00; A61B 5/04

(52) U.S. Cl. 600/459; 600/547

(57) ABSTRACT

Device, system and method of determining an acoustic contact between an ultrasonic transducer and a body subject to ultrasonic energy generated by the transducer. For example, a method of determining an acoustic contact between an ultrasonic transducer and a body subject to ultrasonic energy generated by the transducer may include determining an acoustic contact level between the transducer and the body based on at least one value of at least one parameter related to an electrical impedance at the transducer. Other embodiments are described and claimed.

### FIELD

Some embodiments are related to the field of non-invasive ultrasonic treatment.

### BACKGROUND

An ultrasound-based therapeutic procedure may be used as part of any suitable therapeutic procedure.

In one example, the ultrasound-based therapeutic procedure may be implemented as part of an aesthetic medical treatment, e.g., for fat and/or adipose tissue removal. The ultrasound-based therapeutic procedure may involve a non-invasive treatment, which may be based on the application of focused therapeutic ultrasound energy, which selectively targets and disrupts fat cells, e.g., without damaging neighboring structures. For example, a device, such as a transducer, may be used to deliver focused ultrasound energy to the subcutaneous fat layer. Specific, pre-set ultrasound parameters may ensure that only the fat cells within the treatment area are targeted and that neighboring structures such as blood vessels, nerves and connective tissue remain intact. The ultrasound-based therapeutic procedure may be implemented as part of any other suitable therapeutic treatment.

During the non-invasive ultrasonic procedure it may be desirable to ensure a good and/or minimal level of acoustic contact between the transducer and the body subject to the ultrasonic energy generated by the transducer, e.g., in order to ensure efficient transfer of the ultrasonic energy from the transducer to the body.

### SUMMARY

Some embodiments include, for example, devices, systems and methods of determining an acoustic contact between an ultrasonic transducer and a body subject to ultrasonic energy, for example, therapeutic ultrasonic energy, generated by the transducer.

Some embodiments include, for example, a method of determining an acoustic contact between an ultrasonic transducer and a body subject to ultrasonic energy, for example, therapeutic ultrasonic energy, generated by the transducer. The method may include determining an acoustic contact level between the transducer and the body based on at least one value of at least one parameter related to electrical impedance at the transducer.

In some embodiments, the electrical impedance at the transducer may include a combined electrical impedance of at least the body and the transducer.

In some embodiments, the method may include measuring the value using an impedance measurement device.

In some embodiments, determining the acoustic contact level may include determining the acoustic contact level based on the value of the parameter in response to a time-dependent electrical signal applied to the transducer.

In some embodiments, the method may include sampling the current and voltage of the electrical signal at a sampling frequency of at least twice a frequency of the electrical signal to determine a plurality of current sample values and a plurality of voltage sample values; and determining the value of the parameter based on the plurality of current sample values and the plurality of voltage sample values.

In some embodiments, the time-dependent electrical signal may include at least part of an electrical signal, which is applied to the transducer to generate the ultrasonic energy.

In some embodiments, the time-dependent electrical signal is different than an electrical signal, which is applied to the transducer to generate the ultrasonic energy.

In some embodiments, the method may include applying the time-dependent electrical signal to the transducer before a pulse of a pulsed electrical signal, which is applied to the transducer to generate the ultrasonic energy.

In some embodiments, the method may include selectively providing to the transducer an electrical signal to generate the ultrasonic energy based on the determined acoustic contact level.

In some embodiments, the at least one parameter may include at least one of a module of the impedance, a resistance component of the impedance, a reactance component of the impedance, a capacitance component of the impedance, a ratio between the resistance and reactance components of the impedance, and a phase of the impedance.

In some embodiments, determining the acoustic contact level may include comparing the value of the parameter to at least one value representing at least one level of acoustic contact.

In some embodiments, the method may include updating the value representing the level of acoustic contact based on a detected variation in the value of the parameter.

In some embodiments, determining the acoustic contact level may include selecting between first and second acoustic contact levels.

Some embodiments include an ultrasonic treatment system, which may include an acoustic contact evaluator capable of determining an acoustic contact level between an ultrasonic transducer and a body subject to ultrasonic energy, e.g., therapeutic ultrasonic energy, generated by the transducer based on at least one value of at least one parameter related to an electrical impedance at the transducer.

In some embodiments, the electrical impedance at the transducer may include a combined electrical impedance of at least the body and the transducer.

In some embodiments, the system may include an impedance measurement device to measure the value.

In some embodiments, the acoustic contact evaluator is capable of determining the acoustic contact level based on the value of the parameter in response to a time-dependent electrical signal applied to the transducer.

In some embodiments, the system may include a generator to provide a time-dependent electrical signal to the transducer, wherein the acoustic contact evaluator is capable of determining the acoustic contact level based on the value of the parameter in response to the time-dependent electrical signal.

In some embodiments, the acoustic contact evaluator is to receive a plurality of current sample values and a plurality of voltage sample values corresponding to the time-dependent electrical signal, and to determine the value of the parameter based on the plurality of current sample values and the plurality of voltage sample values.

In some embodiments, the time-dependent electrical signal may include at least part of an electrical signal, which is applied to the transducer to generate the ultrasonic energy.

In some embodiments, the time-dependent electrical signal is different than an electrical signal, which is applied to the transducer to generate the ultrasonic energy.

In some embodiments, the generator is to provide the time-dependent electrical signal to the transducer before a pulse of a pulsed electrical signal, which is applied to the transducer to generate the ultrasonic energy.

In some embodiments, the system may include a controller to control the generator, based on the determined acoustic contact level, to selectively provide the transducer with an electrical signal to generate the ultrasonic energy.

In some embodiments, the at least one parameter may include at least one of a module of the impedance, a resistance component of the impedance, a reactance component of the impedance, a capacitance component of the impedance, a ratio between the resistance and reactance components of the impedance, and a phase of the impedance.

In some embodiments, the acoustic contact evaluator is to determine the acoustic contact level by comparing the value of the parameter to at least one value representing at least one level of acoustic contact.

In some embodiments, the acoustic contact evaluator is capable of updating the value representing the level of acoustic contact based on a detected variation in the value of the parameter.

In some embodiments, the system may include a user interface to provide an indication of the acoustic contact level.

Some embodiments include a computer program product, which may include a computer-useable medium including a computer-readable program, wherein the computer-readable program, when executed on a computer, causes the computer to determine an acoustic contact level between an ultrasonic transducer and a body subject to ultrasonic energy, e.g., therapeutic ultrasonic energy, generated by the transducer based on at least one value of at least one parameter related to an electrical impedance at the transducer.

In some embodiments, the electrical impedance at the transducer may include a combined electrical impedance of at least the body and the transducer.

In some embodiments, the computer-readable program when executed on the computer causes the computer to determine the acoustic contact level based on the value of the parameter in response to a time-dependent electrical signal applied to the transducer.

In some embodiments, the computer-readable program causes the computer to receive a plurality of current sample values and a plurality of voltage sample values corresponding to the time-dependent electrical signal, and to determine the value of the parameter based on the plurality of current sample values and the plurality of voltage sample values.

In some embodiments, the at least one parameter may include at least one of a module of the impedance, a resistance component of the impedance, a reactance component of the impedance, a capacitance component of the impedance, a ratio between the resistance and reactance components of the impedance, and a phase of the impedance.

In some embodiments, the computer-readable program causes the computer to determine the acoustic contact level by comparing the value of the parameter to at least one value representing at least one level of acoustic contact.

In some embodiments, the computer-readable program causes the computer to provide to a user an indication of the acoustic contact level.

Some embodiments may provide other and/or additional benefits and/or advantages.

### BRIEF DESCRIPTION OF THE DRAWINGS

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. The figures are listed below.

FIG. 1 is a schematic block diagram illustration of a non-invasive ultrasonic treatment system, in accordance with some demonstrative embodiments;

FIG. 2A is a schematic illustration of a graph depicting the values of a reactance component of an impedance at a first transducer subject to an electrical signal having a frequency of 200 kHz as a function of an Acoustic Contact (AC) level, in accordance with some demonstrative embodiments;

FIG. 2B is a schematic illustration of first and second graphs depicting the values of a resistance for the first transducer subject to an electrical signal having a frequency of 188 kHz, and the values of the resistance for a second transducer subject to an electrical signal having a frequency of 214 kHz; respectively, as a function of the AC level, in accordance with some demonstrative embodiments;

FIG. 2C is a schematic illustration of first and second graphs depicting the values of the resistance for the first transducer subject to an electrical signal having a frequency of 10 kHz, and the values of the resistance for the second transducer subject to an electrical signal having a frequency of 10 kHz, respectively, as a function of the AC level, in accordance with some demonstrative embodiments;

FIG. 3 is a schematic flow-chart illustration of a method of determining an acoustic contact between an ultrasonic transducer and a body subject to ultrasonic energy generated by the transducer, in accordance with some demonstrative embodiments;

FIG. 4 is a schematic illustration of a demonstrative treatment scheme of a patient by a caregiver, in accordance with some demonstrative embodiments; and

FIG. 5 is a schematic illustration of a transducer, in accordance with some demonstrative embodiments.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

Discussions herein utilizing terms such as, for example, “processing,” “computing”, “calculating”, “determining”, “establishing”, “analyzing”, “checking”, or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

The terms “plurality” and “a plurality” as used herein include, for example, “multiple” or “two or more”. For example, “a plurality of items” includes two or more items.

Some embodiments may include apparatuses for performing the operations herein. These apparatuses may be specially constructed for the desired purposes, or they may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, a Dynamic RAM (DRAM), a Synchronous DRAM (SD-RAM), a Flash memory, a volatile memory, a non-volatile memory, a cache memory, a buffer, a short term memory unit, a long term memory unit, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method. The desired structure for a variety of these systems will appear from the description below. In addition, some embodiments are not described with reference to any particular programming language. It will be appreciated that a variety of programming languages may be used to implement the teachings described herein. Some non-limiting demonstrative embodiments may be implemented using any suitable embedded system.

Although portions of the discussion herein relate, for demonstrative purposes, to wired links and/or wired communications, some embodiments are not limited in this regard, and may include one or more wired or wireless links, may utilize one or more components of wireless communication, may utilize one or more methods or protocols of wireless communication, or the like. Some embodiments may utilize wired communication and/or wireless communication.

Some embodiments include a transducing system including a transducer capable of transducing vibration energy such as ultrasound energy. The ultrasound energy may be focused and may be used, for example, to selectively target and disrupt fat cells without damaging neighboring tissues in a subject body.

In some non-limiting embodiments, the terms “subject body” and “body” as used herein may include all or any part of a subject body, both internally and/or externally. For example, a “subject body” may include an entire body; body part, such as a limb; an organ, such as for example, a liver; a tissue, such as for example, skin tissue, subcutaneous adipose tissue, blood vessel, nerve tissue and the like; cells, such as, for example, fat cells, blood cells, and the like.

As referred to herein, the terms transducer, transducer unit, transducing unit, therapeutic transducer, and vibration delivery system may interchangeably be used.

As referred to herein, the terms acoustic energy, acoustic waves, ultrasonic energy, ultrasonic waves, vibration energy, and vibration waves, may interchangeably be used.

In some non-limiting embodiments, the term “Acoustic Contact” (AC) as used herein may refer to a level, amount, and/or degree of a physical and/or mechanical contact, coupling and/or connection between a transducer and a body which may be subject to ultrasonic energy, e.g., therapeutic ultrasonic energy, generated by the transducer. The AC between the transducer and the body may represent, for example, a ratio between an amount of the ultrasonic energy generated by the transducer and an amount of the ultrasonic energy received by the body. The AC between the transducer and the body may be expressed, determined, evaluated, calculated and/or measured, for example, relative to one or more AC values, e.g., including one or more predetermined and/or predefined AC values. In one example, the AC between the transducer and the body may be determined relative to a first AC value (“no AC” or “poor AC”) representing a relatively low AC, for example, when the transducer is placed in a substance having a relatively low characteristic acoustic impedance, e.g., 420 Rayls, for example, a gaseous substance, e.g., air; and/or a second AC value (“good AC”) representing a relatively high AC, for example, when the transducer is placed in a substance having a relatively high characteristic acoustic impedance, e.g., 1.5 Mega Rayls, for example, a liquid substance, e.g., water.

Although not limited in this respect, in some embodiments the phrase “body subject to ultrasonic energy generated by a transducer” as used herein may relate to any suitable body, element, or substance, which may be in direct or in indirect contact with the transducer to receive the ultrasonic energy generated by the transducer. For example, the body may be placed, at least partially, in indirect contact with the transducer, to receive the ultrasonic energy generated by the transducer. In one non-limiting example, any suitable intermediate medium, for example, any suitable gel, hydro-gel, water, oil and the like, may be applied between the body and the transducer, e.g., as described below.

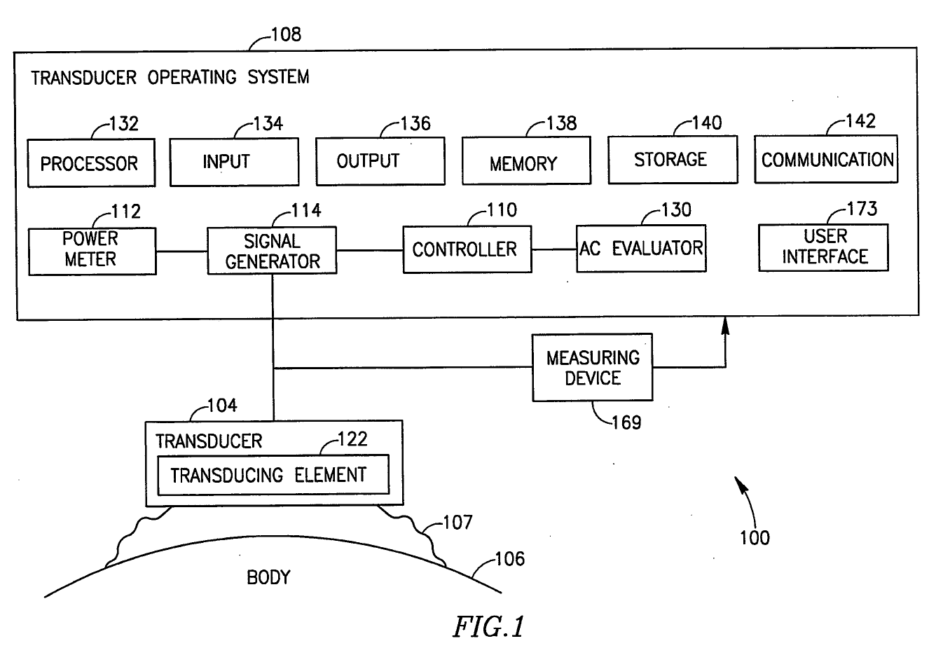

Reference is made to FIG. 1, which schematically illustrates a non-invasive ultrasonic treatment system 100, in accordance with some demonstrative embodiments.

In some non-limiting embodiments, system 100 may be implemented to perform any suitable therapeutic treatment. For example, system 100 may be implemented to deliver focused ultrasound energy to one or more subcutaneous regions, areas and/or layers.

In some non-limiting examples, system 100 may be implemented to remove and/or reduce the number of subcutaneous fat cells and/or volume of adipose tissue resulting, for example, in reshaping of body parts. In other examples, system 100 may be implemented as part of any other therapeutic treatment or procedure, for example, a treatment for tumors ablation.

In some non-limiting embodiments, system 100 may enable performing a non-invasive treatment by applying focused therapeutic ultrasound energy, which selectively targets and disrupts fat cells, e.g., without damaging neighboring structures. For example, system 100 may be implemented to deliver focused ultrasound energy to a subcutaneous fat layer. Specific, pre-set ultrasound parameters may ensure that only fat cells within a treatment area are targeted and that neighboring structures such as blood vessels, nerves and connective tissue remain intact.

In some embodiments, system 100 may include a transducer operating system 108 capable of providing a transducer 104 with, for example, power, energy, fluids, software instruction, control, feedback information, and the like. Transducer 104 may include, for example, transducer 500 as described below with reference to FIG. 5, or any other suitable transducer. Transducer operating system 108 may include one or more subunits that may be independent and/or interconnected and may be individually or commonly controlled, e.g., as described herein.

In some embodiments, transducer operating system 108 may include a controller 110 including any appropriate hardware and/or software that may be used to control and/or coordinate operation of various components of system 100. For example, controller 110 may include electronic circuits, processors, ROM and RAM memories, and the like. Controller 110 may receive and send information from the various components of system 100, e.g., as described herein.

In some embodiments, transducer operating system 108 may include one or more power sources, for example, a signal generator 114 to provide power to transducer 104. In one example, signal generator 114 may provide power to at least one transducing element 122 located within transducer 104. In one non-limiting example, signal generator 114 may provide pulses having a duration of, for example, between about 0.1 and 10 seconds, for example, a duration of between about 0.1 and 8 seconds, for example, a duration of between about 0.1 and 6 seconds, for example, a duration of between about 0.1 and 4 seconds, for example, a duration of between about 0.1 and 3 seconds, for example, a duration of between about 0.1 and 2 seconds, for example, a duration of between about 0.1 and 1 seconds, e.g., a duration of between about 0.1 and 0.5 seconds. In another non-limiting example, signal generator 114 may provide pulses having durations in the order of, for example, 30 minutes, 60 minutes or more, e.g., as part of a treatment for tumors ablation. In some non-limiting embodiments, signal generator 114 may provide the pulses in a continuous (CW) mode, such that transducing element 122 substantially continuously generates ultrasonic energy during substantially the entire duration of the pulse. In some non-limiting embodiments, signal generator 114 may provide the pulses in a burst mode, such that transducing element 122 generates ultrasonic energy in periodic bursts, which may be characterized by repetition frequency and duty cycle (DC). The duty cycle may correspond to a portion of the burst within a period, e.g., a DC of 1%, 5%, 10%, or any other suitable value. In one non-limiting example, the duty cycle may be about 1/20 (5%), and the repetition frequency may be about 25 Hz.

In some embodiments, transducing element 122 may produce vibration energy, such as in the form of acoustic energy. In some embodiments, the acoustic energy produced by transducing element 122 may be focused. In some embodiments, transducing element 122 may include a single transducing element. In other embodiments, transducing element 122 may include a plurality of transducing elements, which may be, for example, individually driven with electrical signals of controlled phase, frequency, amplitude and/or time relationships.

In some embodiments, transducer operating system 108 may also include a power meter 112 to perform power measurement and/or control of signal generator 114.

In some embodiments, the communication between various subunits and elements of transducer operating system 108 and transducer 104 may be performed by any suitable wired connection, e.g., connectors, cables, wires, pipes, and the like; and/or any suitable wireless connection.

In some embodiments, one or more of transducer operating system 108 and/or transducer 104 may include one or more devices, units and/or elements described by U.S. patent application Ser. No. 11/870,521, filed Oct. 11, 2007, and entitled “Coating of polyurethane membrane”, the entire disclosure of which is incorporated herein by reference. In other embodiments, transducer operating system 108 and/or transducer 104 may include any other suitable devices, units and/or elements.

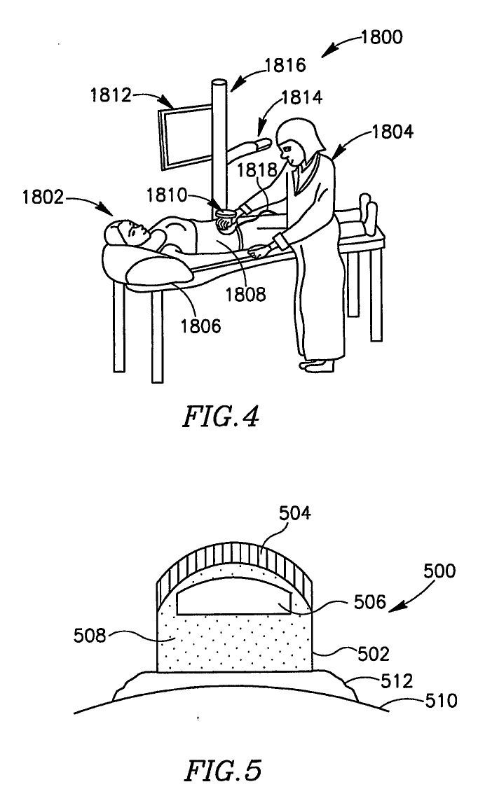

Reference is also made to FIG. 4, which schematically illustrates a demonstrative treatment scheme 1800 of a patient 1802 by a caregiver 1804, in accordance with some demonstrative embodiments. In one non-limiting example, treatment scheme 1800 may be implemented using system 100 (FIG. 1). Caregiver 1804 may be, for example, a physician, a nurse and/or any other person legally and/or physically competent to perform a therapeutic procedure and/or treatment, e.g., a body contouring procedure, involving non-invasive adipose tissue destruction. Patient 1802 optionally lies on a bed 1806 throughout the treatment.

In some embodiments, caregiver 1804 may hold a transducer unit 1810 against an area of a body of patient 1802, e.g., where destruction of adipose tissues is desired. For example, transducer unit 1810 may be held against an abdomen 1808 of patient 1802. Transducer unit 1810 may perform, for example, the functionality of transducer 104 (FIG. 1). For example, transducer unit 1810 may be connected by at least one wire 1818 to one or more elements, for example, a controller, e.g., controller 110 (FIG. 1), and/or to a power source, e.g., signal generator 114 (FIG. 1), of a transducer operating system, e.g., transducer operating system 108 (FIG. 1).

In some embodiments, a user interface is displayed on a monitor 1812, which may be functionally affixed to a rack, such as pillar 1816. For example, monitor 1812 may perform the functionality of an output 136 (FIG. 1) and/or a user interface 173 (FIG. 1), as described herein. A transducer unit 1810 storage ledge 1814 may be provided on pillar 1816 or elsewhere.

The therapeutic procedure and/or treatment, e.g., body contouring, may be performed by emitting one or more ultrasonic pulses from transducer unit 1810 while transducer unit 1810 is held against a certain area of the body of patient 1802. Then, transducer unit 1810 may optionally be re-positioned above one or more additional areas and the emitting is repeated. Each position of transducer unit 1810 may be referred to as a “node”. A single body contouring treatment may include treating a plurality of nodes.

In one non-limiting example, it may be desired and/or required to determine the AC level between transducer unit 1810 and the body of patient 1802. For example, caregiver 1804 may have no other efficient and/or proper way of assuring that the ultrasonic energy generated by transducer unit 1810 is effectively and/or properly received by the body of patient 1802, e.g., as opposed to an operator of an ultrasonic imaging system which may adjust a positioning of an ultrasonic transducer based on a received image.

Referring back to FIG. 1, in some embodiments, transducer operating system 108 may include an AC evaluator 130 capable of determining an AC level between transducer 104 and a body 106 subject to ultrasonic energy generated by transducer 104 based on at least one value of at least one parameter, denoted P, related to an electrical impedance, denoted Z, at transducer 104. The electrical impedance at transducer 104 may include, for example, a combined electrical impedance of at least body 106 and transducer 104, e.g., as described herein.

In some embodiments, body 106 may include any suitable body, element, or substance, which may be in direct or in indirect contact with transducer 104 to receive the ultrasonic energy generated by a transducer. For example, body 106 may be placed, at least partially, in indirect contact with transducer 104, to receive the ultrasonic energy generated by transducer 104. In one non-limiting example, any suitable intermediate medium 107, for example, any suitable gel, hydro-gel, water, oil and the like, may be applied between body 106 and transducer 104, e.g., in order to increase and/or improve the AC level between transducer 104 and body 106. Accordingly, the electrical impedance at transducer 104 may include, for example, a combined electrical impedance of body 106, intermediate medium 107, and transducer 104.

In some demonstrative embodiments, determining the AC level between transducer 104 and body 106 may enable caregiver 1804 (FIG. 4) to determine whether or not the ultrasonic energy generated by transducer 104 is effectively and/or properly received by body 106; and/or to adjust a relative positioning between transducer 104 and/or body 106 based on the determined AC level, for example, in order to improve the AC level between transducer 104 and body 106, e.g., if the determined AC level is not satisfactory.

In some embodiments, the AC level may be affected by one or more parameters related to intermediate medium 107, for example, the amount, purity and/or clarity of intermediate medium 107. For example, an insufficient amount of intermediate medium 107 and/or a contamination of intermediate medium 107, e.g., by particles, dust, hair, trapped air bubbles and the like, may result in a reduced AC level between transducer 104 and body 106.

In some embodiments, caregiver 1804 (FIG. 4) may selectively add, remove and/or replace intermediate medium 107, e.g., during the treatment, for example, based on the determined AC level. In some embodiments, transducer operating system 108 may be capable of automatically applying intermediate medium 107 e.g., during the treatment, for example, based on the determined AC level.

In some embodiments, controller 110 may control signal generator 114, based on the determined AC level, to selectively provide transducer 104 with an electrical signal to generate the ultrasonic energy. For example, controller 110 may control signal generator 114 to provide transducer 104 with the electrical signal to generate the ultrasonic energy, for example, only if the determined AC level is satisfactory, e.g., as described below.

In some embodiments, one or more elements of transducer operating system 108 may be implemented as part of any suitable computing system. For example, transducer operating system 108 may include a processor 132 capable of executing instructions, which may be stored, for example, by a memory 138 and/or a storage 140, resulting in AC evaluator 130, controller 110 and/or user interface 173.

In some demonstrative embodiments, processor 132 includes, for example, a central processing unit (CPU), a digital signal processor (DSP), a microprocessor, a host processor, a controller, a plurality of processors or controllers, a chip, a microchip, one or more circuits, circuitry, a logic unit, an integrated circuit (IC), an application-specific IC (ASIC), or any other suitable multi-purpose or specific processor or controller.

In some demonstrative embodiments, memory 138 includes, for example, a RAM, a ROM, a DRAM, a SD-RAM, a flash memory, a volatile memory, a non-volatile memory, a cache memory, a buffer, a short term memory unit, a long term memory unit, or other suitable memory units or storage units. Storage 140 includes, for example, a hard disk drive, a floppy disk drive, a compact disk (CD) drive, a CD-ROM drive, a digital versatile disk (DVD) drive, or other suitable removable or non-removable storage units.

In some demonstrative embodiments, transducer operating system 108 may also include an input unit 134, an output unit 136, a communication unit 142, and/or any other suitable components. Input unit 134 includes, for example, a keyboard, a keypad, a mouse, a touch-pad, a stylus, a microphone, or other suitable pointing device or input device. Output unit 136 includes, for example, a cathode ray tube (CRT) monitor or display unit, a liquid crystal display (LCD) monitor or display unit, a screen, a monitor, a speaker, or other suitable display unit or output device. Output unit 136 may display, for example, user interface 173, for example, a Graphical User Interface (GUI), to indicate the AC level, e.g., as described below. Communication unit 142 includes, for example, a wired or wireless network interface card (NIC), a wired or wireless modem, a wired or wireless receiver and/or transmitter, a wired or wireless transmitter-receiver and/or transceiver, a radio frequency (RF) communication unit or transceiver, or other units able to transmit and/or receive signals, blocks, frames, transmission streams, packets, messages and/or data. Communication unit 142 may optionally include, or may optionally be associated with, for example, one or more antennas, e.g., a dipole antenna, a monopole antenna, an omni-directional antenna, an end fed antenna, a circularly polarized antenna, a micro-strip antenna, a diversity antenna, or the like.

In some demonstrative embodiments, the components of transducer operating system 108 are enclosed in, for example, a common housing, packaging, or the like, and are interconnected or operably associated using one or more wired or wireless links. In other embodiments, for example, components of transducer operating system 108 may be distributed among multiple or separate devices, may be implemented using a client/server configuration or system, may communicate using remote access methods, or the like.

In some embodiments, AC evaluator 130 may determine the AC level based on the value of the parameter P in response to a time-dependent electrical signal applied to transducer 104. In one example, signal generator 114 may provide a time-dependent electrical signal to transducer 104, e.g., as described herein; and AC evaluator 130 may determine the AC level based on the value of the parameter P in response to the time-dependent electrical signal.

In some embodiments, the time-dependent electrical signal may include at least part of an electrical signal, which is applied to transducer 104 to generate the ultrasonic energy, e.g., therapeutic ultrasonic energy, to be applied to body 106.

In some embodiments, the time-dependent electrical signal may be different than the electrical signal, which is applied to transducer 104 to generate the ultrasonic energy, e.g., therapeutic ultrasonic energy, to be applied to body 106. In one example, generator 114 may provide the time-dependent electrical signal to transducer 104 before a pulse of a pulsed electrical signal, which is applied to transducer 104 to generate the ultrasonic energy to be applied to body 106.

In some embodiments, the time-dependent electrical signal may include a relatively high voltage signal (“high power driving signal”), e.g., of approximately 100 Volts (V). In other embodiments, the time-dependent electrical signal may include a relatively low voltage signal (“small power signal” or “low power signal”), e.g., of approximately 1V. In other embodiments, the time-dependent electrical signal may include a signal of any other suitable voltage.

In some embodiments, the time-dependent electrical signal may include a signal having a rectangular, sinusoidal, triangular, or any other suitable waveform.

In some embodiments, the time-dependent electrical signal may include a signal having any suitable frequency.

In one non-limiting example, the time-dependent electrical signal may include a relatively high voltage signal (“high power signal”), e.g., of approximately 100 Volts (V); having a rectangular, sinusoidal, or triangular waveform; and/or having a frequency of about a first or third harmonic of transducer 104.

In another non-limiting example, the time-dependent electrical signal may include a relatively low voltage signal, e.g., of approximately 1V; having a sinusoidal waveform; and/or having a relatively low frequency, for example, a frequency, e.g., 10 kilo Hertz (kHz), much lower than the first harmonic of transducer 104, a medium frequency, e.g., about the first harmonic of transducer 104, or a relatively high frequency, for example, about the third harmonic of transducer 104.

In other examples, the time-dependent electrical signal may include any other suitable signal, having any suitable voltage, waveform, and/or frequency, for example, a low power signal or a high power signal having a relatively low frequency, for example, a frequency, e.g., 10 kilo Hertz (kHz), much lower than the first harmonic of transducer 104, a medium frequency, e.g., about the first harmonic of transducer 104, or a relatively high frequency, for example, about the third harmonic of transducer 104.

In some embodiments, system 100 may include a measuring device 169 to provide AC evaluator 130 with one or more measurements and/or samples corresponding to transducer 104, e.g., as described in detail below.

In some embodiments, measuring device 169 may sample a plurality of current sample values and a plurality of voltage sample values corresponding to the time-dependent electrical signal, e.g., as described below. AC evaluator 130 may receive from measuring device 169 the plurality of current sample values and the plurality of voltage sample values; and may determine the value of the at least one parameter P based on the plurality of current sample values and the plurality of voltage sample values, e.g., as described below.

In some embodiments, measuring device 169 may include any suitable impedance measurement device to measure the at least one value of the at least one parameter P related to the electrical impedance at transducer 104, e.g., in response to the time-dependent electrical signal. In one non-limiting example, measurement device 169 may include the Hioki 3532-50 LCR HiTESTER available from HIOKI E.E. Corporation, 81 Koizumi Ueda, Nagano 386-1192 Japan. In other examples, measurement device 169 may include any other suitable device.

In some embodiments, the at least one parameter P related to the electrical impedance at transducer 104 may include at least one of a module (“magnitude”), denoted |Z|, of the impedance; a resistance component, denoted R, of the impedance; a reactance component, denoted X, of the impedance; a capacitance component, denoted Cs, of the impedance, a ratio between the resistance and reactance components of the impedance, a phase, denoted θ, of the impedance, and/or any other suitable parameter.

In some non-limiting embodiments, signal generator 114 may provide to transducer 104, for example, an electrical signal, e.g., a sinusoidal signal or any other signal, having a substantially fixed frequency, denoted f₀, and having substantially constant voltage amplitude. Measuring device 169 may be capable of performing voltage and current sampling at substantially constant sampling frequency, denotedFₛ. For example, the sampling frequency may be determined as follows:

Fₛ=D×f₀ (1)

wherein D denotes a predefined integer, for example, equal to or greater than 2.

In some embodiments, measuring device 169 may perform the voltage and current sampling during k periods, wherein k denotes a predefined integer, which is equal to or greater than 1. Accordingly, measuring device 169 may sample N=D×k voltage sample values and N current sample values, which may be represented by two respective N-point arrays, denoted {Vₙ}ₙ₌₀^{N-1} and {Iₙ}ₙ₌₀^{N-1}, respectively.

In some embodiments, the integers D and k may be predefined, for example, based on one or more characteristics of signal generator 114, transducer 104 and/or measuring device 169. In one non-limiting example, measuring device 169 may sample 10 samples per period (D=10), over 20 periods (k=20), resulting in two 200-points arrays (N=200) of the voltage and current samples, respectively.

In some embodiments, the value of the at least one parameter P related to the complex impedance Z at frequency f₀ at transducer 104 may be determined based on the arrays {Vₙ}n=0^{N-1} and {Iₙ}ₙ₌₀^{N-1}, e.g., as described below.

In some embodiments, real and imaginary voltage components, denoted Vᵣ and Vᵢ, respectively, may be determined based on the array {Iₙ}ₙ₌₀⁻¹, for example, as follows:

$$\begin{matrix}{V}_{r} = \frac{1}{N} {\sum }_{n = 0}^{N - 1} {V}_{n} \cdot cos ( \frac{2 π \cdot {f}_{0} \cdot n}{{F}_{s}} ) & ( 2 ) \\ {V}_{i} = \frac{1}{N} {\sum }_{n = 0}^{N - 1} {V}_{n} \cdot sin ( \frac{2 π \cdot {f}_{0} \cdot n}{{F}_{s}} ) & ( 3 )\end{matrix}$$

A magnitude of the voltage, denoted V, may be determined, for example, as follows:

V=\sqrt{Vᵣ²+Vᵢ²} (4)

In some embodiments, real and imaginary voltage components, denoted Iᵣ and Iᵢ, respectively, may be determined based on the array {Iₙ}ₙ₌₀^{N-1}, for example, as follows:

$$\begin{matrix}{I}_{r} = \frac{1}{N} {\sum }_{n = 0}^{N - 1} {I}_{n} \cdot cos ( \frac{2 π \cdot {f}_{0} \cdot n}{{F}_{s}} ) & ( 5 ) \\ {I}_{i} = \frac{1}{N} {\sum }_{n = 0}^{N - 1} {I}_{n} \cdot sin ( \frac{2 π \cdot {f}_{0} \cdot n}{{F}_{s}} ) & ( 6 )\end{matrix}$$

A magnitude of the current, denoted I, may be determined, for example, as follows:

I=\sqrt{Iᵣ²+Iᵢ²} (7)

In some embodiments, the impedance Z may be represented in the following complex Cartesian form:

Z=R+iX (8)

or in the following polar form:

Z=|Z|e^{iθ} (9)

In some embodiments, the module |Z| of the impedance Z may be determined as follows, e.g., based on Ohm's law:

|Z|=V/I (10)

The phase θ of the impedance Z may be determined as follows:

$$θ = {cos}^{- 1} ( \frac{{V}_{r} \cdot {I}_{r} + {V}_{i} \cdot {I}_{i}}{V \cdot I} ) \qquad ( 11 )$$

The resistance component R of the impedance Z may be determined as follows:

Re{z}=|Z|·cos θ≡R (12)

The reactance component X of the impedance Z may be determined as follows:

Im{Z}=|Z|·sin θ≡X (13)

Although some non-limiting embodiments are described herein with relation to determining the at least one value of the parameter P in response to a sinusoidal electrical signal, other embodiments may include determining the at least one value of the at least one parameter related to the electrical impedance at transducer 104 in response to any other suitable electrical signal, e.g., a rectangular signal or any other signal.

In some embodiments, AC evaluator 130 may determine the AC level between transducer 104 and body 106 by comparing the value of the at least one parameter to at least one value representing at least one AC level, e.g., as described below.

In some embodiments, AC evaluator 130 may determine the AC level between transducer 104 and body 106, for example, relative to one or more AC values, e.g., including one or more predetermined and/or predefined AC values. In one example, AC evaluator 130 may determine the AC between transducer 104 and body 106 relative to a first AC value (“no AC” or “poor AC”) representing a relatively low AC, for example, when transducer 104 is placed in a substance having a relatively low characteristic acoustic impedance, e.g., 420 Rayls, for example, a gaseous substance, e.g., air; and/or a second AC value (“good AC”) representing a relatively high AC, for example, when transducer 104 is placed in a substance having a relatively high characteristic acoustic impedance, e.g., 1.5 Mega Rayls, for example, a liquid substance, e.g., water.

In some embodiments, values relative to the poor AC level (“the poor AC values”) of the at least one parameter P, e.g., the module |Z|, the resistance R, the capacitance Cs, and/or the reactance X, may be determined at one or more frequencies, e.g., by applying to transducer 104 the plurality of frequencies while maintaining a relatively low AC level, for example, by placing transducer 104 in a substance having a relatively low characteristic acoustic impedance, for example, a gaseous substance, e.g., air.

In some embodiments, values relative to the good AC level (“the good AC values”) of the at least one parameter P, e.g., the module |Z|, the resistance R, the capacitance Cs, and/or the reactance X, may be determined at the one or more frequencies, e.g., by applying to transducer 104 the plurality of frequencies while maintaining a relatively good AC level, for example, by placing transducer 104 in a substance having a relatively high characteristic acoustic impedance, for example, a liquid substance, e.g., water.

In some embodiments, AC evaluator 130 may determine the AC level between transducer 104 and body 106 in response to an electric signal of a certain frequency, for example, by comparing a current value of the parameter P to the good AC value and/or the poor AC value corresponding to the certain frequency.

In one non-limiting example, AC evaluator 130 may determine the AC level between transducer 104 and body 106 is poor, e.g., if:

—Pa−Pgood|>d1 (14)

wherein Pa denotes the current value of the parameter P when transducer 104 is subject to an electrical signal of a certain frequency, wherein Pgood denotes a value of the parameter P corresponding to the good AC level, and wherein d1 denotes a predefined interval.

In another non-limiting example, AC evaluator 130 may determine the AC level between transducer 104 and body 106 is poor, e.g., if:

|Pa−Ppoor|<d2 (15)

wherein d2 denotes a predefined interval.

In another non-limiting example, AC evaluator 130 may determine the AC level between transducer 104 and body 106 is poor, e.g., if:

|Pa−Pgood|>|Pa−Ppoor| (16)

In one example, AC evaluator 130 may select between the poor and good AC levels, e.g., according to Equations 14, 15, and/or 16. For example, AC evaluator 130 may determine the AC level between transducer 104 and body 106 is poor if a predefined equation of Equations 14-16 is satisfied; and determine the AC level between transducer 104 and body 106 is good if the predefined equation is not satisfied

In other examples, AC evaluator 130 may determine the AC level between transducer 104 and body 106 by applying any other suitable criterion to one or more of the parameters related to the impedance Z, alone or in combination.

In some embodiments, transducer operating system 108 may also include user interface 173 to provide the user of system 100 with an indication of the determined AC level between transducer 104 and body 106. User interface 173 may include, for example, a graphical and/or textual indicator to indicate the AC level to the user of system 100.

In one example, user interface 173 may provide an absolute indication of the determined AC level between transducer 104 and body 106, for example, “good AC”, or “poor AC”, e.g., according to Equations 1, 15 and/or 16.

In another example, user interface 173 may provide a dynamic and/or relative indication of the determined AC level, e.g., by displaying the determined AC level relative to a scale of AC levels. For example, user interface 173 may graphically display a scale having a first end corresponding to the poor AC level and a second end corresponding to the good AC level; and an indicator to point to a location along the scale representing the determined AC level between transducer 104 and body 106 relative to the good and poor AC levels. In other examples, user interface 173 may provide any other suitable indication of the determined AC level between transducer 104 and body 106.

In some embodiments, AC evaluator 130 may be capable of updating the good AC and/or poor AC values based on a detected variation in the value of the at least one parameter P, e.g., as part of a calibration process.

In one example, AC evaluator 130 may automatically detect a variation in the value of the parameter P corresponding to the good AC and/or poor AC levels, and automatically update the value of the parameter P corresponding to the good AC and/or poor AC levels based on the detected variation. For example, AC evaluator 130 may update the good AC values and/or the poor AC values corresponding to a first signal type, e.g., the high power driving signal, based on determined AC values corresponding to a second signal type, e.g., the small power signal, provided between bursts of the high power driving signal.

In another example, the user of system 100 may manually perform a calibration operation to manually update the value of the parameter P corresponding to the good AC and/or poor AC levels, e.g., by placing transducer 104 in water and/or air in order to determine updated values of the parameter P corresponding to the good AC and/or poor AC levels, respectively.

In some embodiments, controller 110 may receive an indication of the determined AC level from evaluator 130. Controller 110 may control signal generator 114, based on the determined AC level, to selectively provide transducer 104 with an electrical signal to generate the ultrasonic energy. For example, controller 110 may control signal generator 114 to provide transducer 104 with the electrical signal to generate the ultrasonic energy, for example, only if the determined AC level between transducer 104 and body 106 is good, e.g., as described above. For example, controller 110 may cause signal generator 114 not to provide transducer 104 with the electrical signal to generate the ultrasonic energy, for example, if the determined AC level between transducer 104 and body 106 is poor, e.g., as described above.

Following are non-limiting demonstrative values of the module |Z|, the resistance R, the capacitance Cs, and the reactance X corresponding to a first transducer of a first type, having a resonance frequency of 188 kHz, when placed either in air (poor AC) or in water (good AC) in response to electrical signals having frequencies of 200 kHz, 188 kHz, 10 kHz, and 600 dHz. The first transducer may include, for example, the AAC-20000 transducer available from Ultrashape Ltd., New Industrial Park, Yokneam, Israel (“Ultrashape Ltd.”).

TABLE 1

| Frequency | Good AC? | \|Z\| | R | Cs | X |
|---|---|---|---|---|---|
| (kHz) | (In water) | (ohm) | (ohm) | (nanoFarad) | (ohm) |
| 200 | Yes | 40.3 | 40.3 | 880 | 0.9 |
| 200 | No | 35.4 | 35 | 150 | 5.4 |
| 200 | Yes | 39.3 | 39.1 | 1100 | 0.9 |
| 200 | No | 35.5 | 35.2 | 175 | 4.5 |
| 188 | Yes | 31.8 | 19.5 | 33.6 | 25.3 |
| 188 | No | 30.5 | 16.4 | 32.8 | 25.7 |
| 188 | Yes | 32.8 | 19.4 | 31.8 | 26.7 |
| 188 | No | 30.3 | 16.5 | 33.3 | 25.4 |
| 10 | Yes | 2200 | 25 | 7.2 | 2200 |
| 10 | No | 2200 | 12.7 | 7.2 | 2200 |
| 10 | Yes | 2200 | 25 | 7.2 | 2200 |
| 10 | No | 2200 | 12.5 | 7.2 | 2200 |
| 600 | Yes | 106.4 | 1.9 | 2.5 | 106.4 |
| 600 | No | 106.6 | 2 | 2.5 | 106.6 |
| 600 | Yes | 106.6 | 1.9 | 2.5 | 106.6 |
| 600 | No | 106.6 | 1.98 | 2.5 | 106.6 |

Following are non-limiting demonstrative values of the module |Z|, the resistance R, the capacitance Cs, and the reactance X corresponding to a second transducer of a second type, e.g., a third harmonic transducer, when placed either in air (poor AC) or in water (good AC) in response to electrical signals having frequencies of 214 kHz (first harmonic), 700 kHz (third harmonic), and 10 kHz.

TABLE 2

| Frequency | Good AC? | | R | Cs | X |
|---|---|---|---|---|---|
| (kHz) | (In water) | \|Z\| (ohm) | (ohm) | (nanoFarad) | (ohm) |
| 214 | Yes | 36 | 31 | 44 | 16 |
| 214 | No | 28 | 24 | 48 | 15 |
| 214 | Yes | 36 | 32 | 43 | 16 |
| 214 | No | 29 | 24 | 47 | 15 |
| 700 | Yes | 2.59 | 2.59 | 140 | 554 |
| 700 | No | 2.76 | 2.651 | 302 | 751 |
| 700 | Yes | 2.59 | 2.53 | 410 | 554 |
| 700 | No | 2.74 | 2.65 | 300 | 758 |
| 10 | Yes | 1.67 | 61.6 | 9.5 | 1.66 |
| 10 | No | 1.67 | 28 | 9.5 | 1.67 |
| 10 | Yes | 1.65 | 61.4 | 9.59 | 1.65 |
| 10 | No | 1.67 | 28.26 | 9.5 | 1.67 |

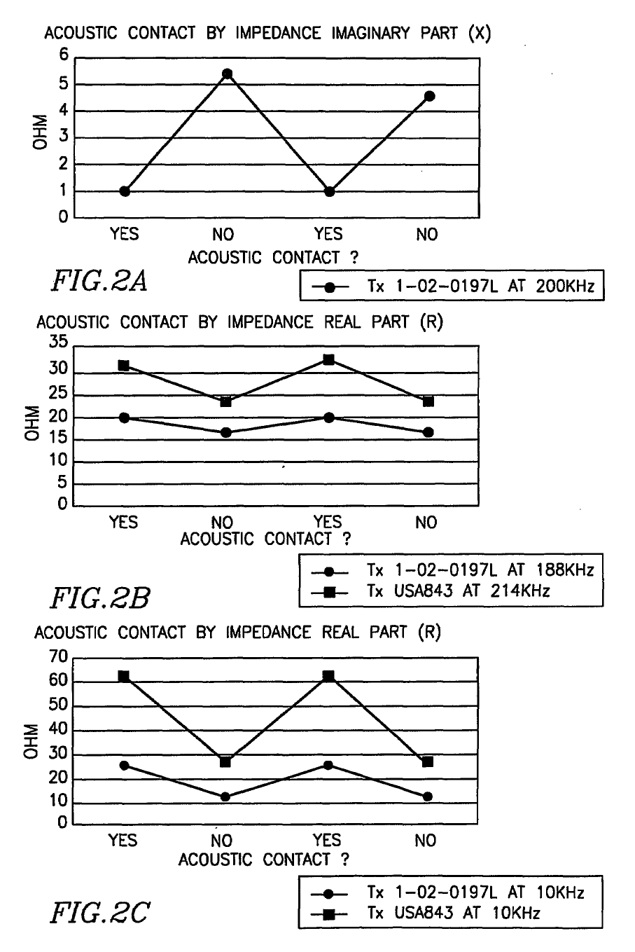

FIG. 2A schematically illustrates a graph depicting the values of the reactance component X for the first transducer (Table 1) at the frequency of 200 kHz as a function of the AC level. As shown in FIG. 2A, a value, e.g., about 1 ohm, of the reactance component X corresponding to the good AC level at the frequency of 200 kHz may be lower than a value, e.g., between 4 and 5 ohms, of the reactance component X corresponding to the poor AC level at the frequency of 200 kHz.

FIG. 2B schematically illustrates first and second graphs depicting the values of the resistance R for the first transducer (Table 1) at the resonance frequency of 188 kHz, and the values of the resistance R for the second transducer (Table 2) at the first harmonic frequency of 214 kHz, respectively, as a function of the AC level. As shown in FIG. 2B, a value, e.g., about 20 ohms, of the resistance R corresponding to the good AC level at the frequency of 188 kHz may be greater than a value, e.g., about 15 ohms, of the resistance R corresponding to the poor AC level at the frequency of 188 kHz. As further shown in FIG. 2B, a value, e.g., about 30 ohms, of the resistance R corresponding to the good AC level at the frequency of 214 kHz may be greater than a value, e.g., about 25 ohms, of the resistance R corresponding to the poor AC level at the frequency of 214 kHz.

FIG. 2C schematically illustrates first and second graphs depicting the values of the resistance R for the first transducer (Table 1) at the frequency of 10 kHz, and the values of the resistance R for the second transducer (Table 2) at the frequency of 10 kHz, respectively, as a function of the AC level. As shown in FIG. 2C, a value, e.g., about 25 ohms, of the resistance R corresponding to the good AC level at the frequency of 10 kHz for the first transducer may be greater than a value, e.g., about 10 ohms, of the resistance R corresponding to the poor AC level at the frequency of 10 kHz. As further shown in FIG. 2B, a value, e.g., about 60 ohms, of the resistance R corresponding to the good AC level at the frequency of 10 kHz for the second transducer may be greater than a value, e.g., about 30 ohms, of the resistance R corresponding to the poor AC level at the frequency of 10 kHz.

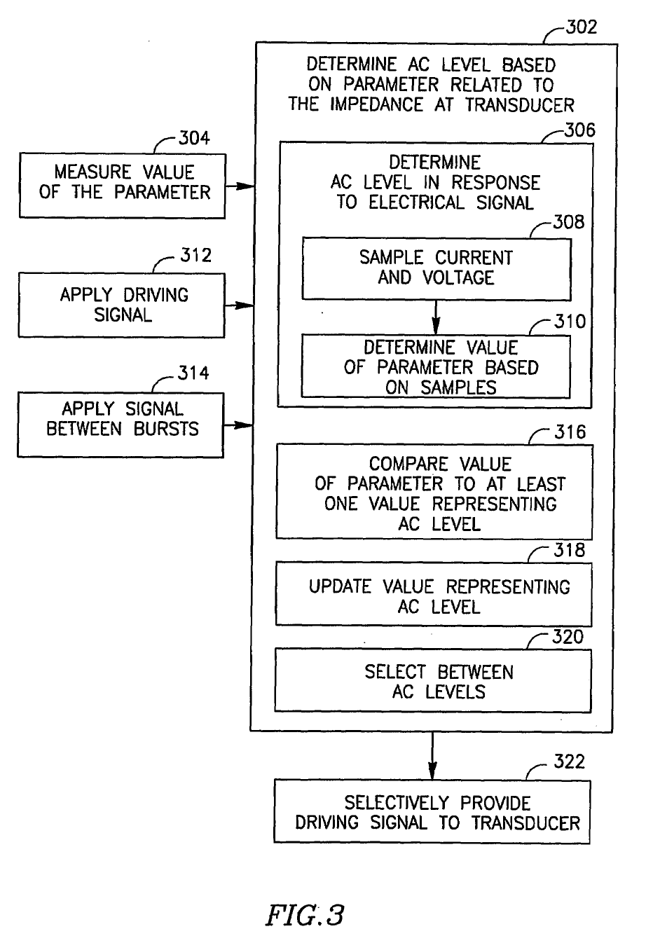

Reference is made to FIG. 3, which schematically illustrates a method of determining an acoustic contact between an ultrasonic transducer and a body subject to ultrasonic energy, e.g., therapeutic ultrasonic energy, generated by the transducer, in accordance with some demonstrative embodiments. In some non-limiting embodiments, one or more operations of the method of FIG. 3 may be implemented, for example, by one or more elements of a non-invasive ultrasonic treatment system, e.g., system 100 (FIG. 1), for example, to determine an AC between transducer 104 (FIG. 1) and body 106 (FIG. 1).

As indicated at block 302 the method may include determining an AC level between the transducer and the body based on at least one value of at least one parameter related to electrical impedance at the transducer. For example, AC evaluator 130 (FIG. 1) may determine the AC level between transducer 104 (FIG. 1) and body 106 (FIG. 1) based on the at least one parameter P, as described above.

Determining the AC level between the transducer and the body may include determining a combined electrical impedance of at least the body and the transducer, e.g., as described above.

As indicated at block 304, the method may include measuring the value of the at least one parameter using an impedance measurement device, e.g., using the Hioki 3532-50 LCR HiTESTER device as described above.

As indicated at block 306, determining the AC level may include determining the AC level based on the value of the parameter in response to a time-dependent electrical signal applied to the transducer.

In one example, the method may include sampling the current and voltage of the electrical signal at a sampling frequency of at least twice a frequency of the electrical signal to determine a plurality of current sample values and a plurality of voltage sample values, as indicated at block 308; and determining the value of the parameter based on the plurality of current sample values and the plurality of voltage sample values, as indicated at block 310. For example, the value of the at least one parameter P may be determined using one or more of Equations 1-13, as described above.

As indicated at block 312, the time-dependent electrical signal may include at least part of an electrical signal, which is applied to the transducer to generate the ultrasonic energy, e.g., as described above.

In some embodiments, the time-dependent electrical signal is different than an electrical signal, which is applied to the transducer to generate the ultrasonic energy, e.g., as described above. For example, as indicated at block 314, the method may include applying the time-dependent electrical signal to the transducer before a pulse of a pulsed electrical signal, which is applied to the transducer to generate the ultrasonic energy, e.g., therapeutic ultrasonic energy.

As indicated at block 316, determining the AC level may include comparing the value of the parameter to at least one value representing at least one level of AC. For example, AC evaluator 130 (FIG. 1) may determine the AC level between transducer 104 and body 106 by comparing the determined value of the at least one parameter P to one or more good AC values and/or poor AC values, e.g., as described above.

As indicated at block 318, the method may include updating the value representing the level of AC based on a detected variation in the value of the parameter. For example, AC evaluator 130 (FIG. 1) may update one or more of the good AC values and/or poor AC values, e.g., as described above.

As indicated at block 320, determining the AC level may include selecting between first and second acoustic contact levels. For example, AC evaluator 130 (FIG. 1) may select between the good and poor AC levels, e.g., as described above.

As indicated at block 322, the method may include selectively providing to the transducer an electrical signal to generate the ultrasonic energy, e.g., therapeutic ultrasonic energy, based on the determined AC level. For example, controller 110 (FIG. 1) may control signal generator 114 (FIG. 1), based on the determined AC level, to selectively provide transducer 104 (FIG. 1) with an electrical signal to generate the ultrasonic energy.

Reference is made to FIG. 5, which schematically illustrates a transducer 500, in accordance with some demonstrative embodiments. In some non-limiting examples, transducer 500 may perform the functionality of transducer 104 (FIG. 1).

In some embodiments, transducer 500 may include at least one transducing element 506, for example, at least one suitable radiating element or construction, to generate ultrasonic energy to be applied to a body 510. Transducer element 506 may be located, for example, within a housing 502, which may be formed of any suitable material and/or have any suitable shape and/or configuration.

Transducer 500 may also include an internal medium 508, e.g., oil, which may surround transducing element 506; and/or a reflector/absorber 504 capable of selectively reflecting/absorbing waves generated by transducing element 506 towards body 510.

Any suitable intermediate medium 512, for example, any suitable gel, water, oil and the like, may be applied between body 510 and transducer 500, e.g., in order to increase the AC level between transducer 500 and body 510.

Some embodiments, for example, may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

Functions, operations, components and/or features described herein with reference to one or more embodiments, may be combined with, or may be utilized in combination with, one or more other functions, operations, components and/or features described herein with reference to one or more other embodiments, or vice versa.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A method of determining an acoustic contact between an ultrasonic transducer and a body subject to ultrasonic energy generated by said transducer, the method comprising:
determining an acoustic contact level between said transducer and said body based on at least one value of at least one parameter related to an electrical impedance at said transducer.

2. The method of claim 1, wherein the electrical impedance at said transducer comprises a combined electrical impedance of at least said body and said transducer.

3. The method of claim 1 comprising measuring said value using an impedance measurement device.

4. The method of claim 1, wherein determining said acoustic contact level comprises determining said acoustic contact level based on the value of said parameter in response to a time-dependent electrical signal applied to said transducer.

5. The method of claim 4 comprising:
sampling the current and voltage of said electrical signal at a sampling frequency of at least twice a frequency of said electrical signal to determine a plurality of current sample values and a plurality of voltage sample values; and
determining the value of said parameter based on said plurality of current sample values and said plurality of voltage sample values.

6. The method of claim 4, wherein said time-dependent electrical signal comprises at least part of an electrical signal, which is applied to said transducer to generate said ultrasonic energy.

7. The method of claim 4, wherein said time-dependent electrical signal is different than an electrical signal, which is applied to said transducer to generate said ultrasonic energy.

8. The method of claim 4 comprising applying said time-dependent electrical signal to said transducer before a pulse of a pulsed electrical signal, which is applied to said transducer to generate said ultrasonic energy.

9. The method of claim 1 comprising selectively providing to said transducer an electrical signal to generate said ultrasonic energy based on said acoustic contact level.

10. The method of claim 1, wherein said at least one parameter comprises at least one of a module of said impedance, a resistance component of said impedance, a reactance component of said impedance, a capacitance component of said impedance, a ratio between the resistance and reactance components of said impedance, and a phase of said impedance.

11. The method of claim 1, wherein determining said acoustic contact level comprises comparing the value of said parameter to at least one value representing at least one level of acoustic contact.

12. The method of claim 1 comprising updating the value representing said level of acoustic contact based on a detected variation in the value of said parameter.

13. The method of claim 1, wherein determining said acoustic contact level comprises selecting between first and second acoustic contact levels.

14. An ultrasonic treatment system, the system comprising:
an acoustic contact evaluator capable of determining an acoustic contact level between an ultrasonic transducer and a body subject to ultrasonic energy generated by said transducer based on at least one value of at least one parameter related to an electrical impedance at said transducer.

15. The system of claim 14, wherein the electrical impedance at said transducer comprises a combined electrical impedance of at least said body and said transducer.

16. The system of claim 14 comprising an impedance measurement device to measure said value.

17. The system of claim 14, wherein said acoustic contact evaluator is capable of determining said acoustic contact level based on the value of said parameter in response to a time-dependent electrical signal applied to said transducer.

18. The system of claim 14 comprising a generator to provide a time-dependent electrical signal to said transducer, and wherein said acoustic contact evaluator is capable of determining said acoustic contact level based on the value of said parameter in response to said time-dependent electrical signal.

19. The system of claim 18, wherein said acoustic contact evaluator is to receive a plurality of current sample values and a plurality of voltage sample values corresponding to said time-dependent electrical signal, and to determine the value of said parameter based on said plurality of current sample values and said plurality of voltage sample values.

20. The system of claim 18, wherein said time-dependent electrical signal comprises at least part of an electrical signal, which is applied to said transducer to generate said ultrasonic energy.

21. The system of claim 18, wherein said time-dependent electrical signal is different than an electrical signal, which is applied to said transducer to generate said ultrasonic energy.

22. The system of claim 18, wherein said generator is to provide said time-dependent electrical signal to said transducer before a pulse of a pulsed electrical signal, which is applied to said transducer to generate said ultrasonic energy.

23. The system of claim 18, comprising a controller to control said generator based on the acoustic contact level to selectively provide said transducer with an electrical signal to generate the ultrasonic energy

24. The system of claim 14, wherein said at least one parameter comprises at least one of a module of said impedance, a resistance component of said impedance, a reactance component of said impedance, a capacitance component of said impedance, a ratio between the resistance and reactance components of said impedance, and a phase of said impedance.

25. The system of claim 14, wherein said acoustic contact evaluator is to determine said acoustic contact level by comparing the value of said parameter to at least one value representing at least one level of acoustic contact.

26. The system of claim 25, wherein said acoustic contact evaluator is capable of updating the value representing said level of acoustic contact based on a detected variation in the value of said parameter.

27. The system of claim 14 comprising a user interface to provide an indication of said acoustic contact level.

28. A computer program product comprising a computer-useable medium including a computer-readable program, wherein the computer-readable program when executed on a computer causes the computer to:
determine an acoustic contact level between an ultrasonic transducer and a body subject to ultrasonic energy generated by said transducer based on at least one value of at least one parameter related to an electrical impedance at said transducer.

29. The computer program product of claim 28, wherein the electrical impedance at said transducer comprises a combined electrical impedance of at least said body and said transducer.

30. The computer program product of claim 28, wherein the computer-readable program, when executed on the computer, causes the computer to determine said acoustic contact level based on the value of said parameter in response to a time-dependent electrical signal applied to said transducer.

31. The computer program product of claim 30, wherein the computer-readable program causes the computer to receive a plurality of current sample values and a plurality of voltage sample values corresponding to said time-dependent electrical signal, and to determine the value of said parameter based on said plurality of current sample values and said plurality of voltage sample values.

32. The computer program product of claim 28, wherein said at least one parameter comprises at least one of a module of said impedance, a resistance component of said impedance, a reactance component of said impedance, a capacitance component of said impedance, a ratio between the resistance and reactance components of said impedance, and a phase of said impedance.

33. The computer program product of claim 28, wherein the computer-readable program causes the computer to determine said acoustic contact level by comparing the value of said parameter to at least one value representing at least one level of acoustic contact.

34. The computer program product of claim 28, wherein the computer-readable program causes the computer to provide to a user an indication of said acoustic contact level.
